(19) United States
(12) Patent Application Publication
(10) Pub. No.: US 2018/0246434 A1
(43) Pub. Date: Aug. 30, 2018

(54) TONER

(71) Applicant: CANON KABUSHIKI KAISHA, Tokyo (JP)

(72) Inventors: Ichiro Kanno, Kashiwa-shi (JP); Takeshi Hashimoto, Moriya-shi (JP); Nozomu Komatsu, Toride-shi (JP); Masayuki Hama, Toride-shi (JP); Hitoshi Sano, Matsudo-shi (JP); Yuto Onozaki, Saitama-shi (JP); Takakuni Kobori, Toride-shi (JP); Hiroyuki Fujikawa, Yokohama-shi (JP)

(21) Appl. No.: 15/902,910

(22) Filed: Feb. 22, 2018

(30) Foreign Application Priority Data
Feb. 27, 2017 (JP) 2017-035395

(51) Int. Cl. G03G 9/087; G03G 9/09; G03G 9/097; C07C 245/08

(52) U.S. Cl. CPC G03G 9/08728; C07C 245/08; G03G 9/09783; G03G 9/092

(57) ABSTRACT

A toner comprises toner particles each containing a binder resin and a crystalline compound represented by a specific chemical formula. The crystalline compound. exhibits a CuKα X-ray diffraction spectrum having a diffraction peak at a Bragg angle 2θ±0.2 in the range of 4.0° to 5.0°. The diffraction peak has a half-value width of 0.7° or more.

### BACKGROUND OF THE INVENTION

### Field of the Invention

The present disclosure relates to a toner used in an electrophotographic image forming apparatus.

### Description of the Related Art

In general, an electrophotographic image forming apparatus forms an electrostatic latent image on a photosensitive member, develops the electrostatic latent image with a toner into a toner image, and transfers the toner image to a recording medium, such as a paper sheet. The transferred toner image is subsequently fixed to the recording medium by heating and/or pressing with a fixing device to yield a finished image.

For forming a full color image, colors are reproduced by using, typically, three primary color toners of chromatic color toners, that is, a yellow toner, a magenta toner, and a cyan toner, or four color toners constituted of the three primary colors and an achromatic black toner.

In particular, the magenta toner, as well as the yellow toner, is important in reproducing red, to which human beings are visually sensitive. Also, the magenta. toner, as well as the cyan toner, is important in reproducing blue, which is frequently used as a business color.

Various pigments are devised for the magenta toner. Among these are often used insoluble azo pigments and lake pigments produced by a reaction of a soluble azo pigment with a metal compound for laking. These pigments exhibit high tinting strength.

Although insoluble azo pigments and lake pigments have high tinting strength, they are highly crystalline and their crystals are hard and large. Accordingly, these pigments are difficult to disperse into a toner particle. Accordingly, toner particles using an insoluble azo pigment or a lake pigment are likely to be unstable in chargeability, to cause fogging, and to change in color.

Japanese Patent Laid-Open No. 2006-267741 discloses a magenta toner using a quinacridone pigment and an azo-based naphthol pigment in combination.

Japanese Patent Laid-Open Nos. 2014-174527 and 2015-180925 disclose toners using monoazo-based naphthol pigment.

Unfortunately, many of the known magenta toner pigments do not have high tinting strength nor charging stability at the same time, thus being required to be improved so as to have both high tinting strength and charging stability and keep image density stable.

### SUMMARY OF THE INVENTION

The present disclosure provides a toner having a high tinting strength, charging stability, and color stability.

Accordingly, there is provided a toner comprising toner particles each containing a binder resin and a crystalline compound exhibiting a CuKα X-ray diffraction spectrum having a diffraction peak at a Bragg angle 2θ (±0.2) in the range of 4.0° to 5.0°. The diffraction peak has a half-value width of 0.7° or more. The crystalline compound is represented by the following formula (1)

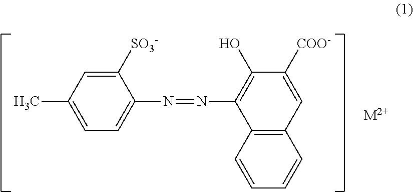

wherein M represents an atom selected from the group consisting of barium, strontium, calcium, and manganese.

The toner can exhibit a high tinting strength, charging stability, and color stability.

Further features will become apparent from the following description of exemplary embodiments.

### DESCRIPTION OF THE EMBODIMENTS

The toner according to an embodiment of the present disclosure comprises toner particles each containing a binder resin and a crystalline compound expressed by the following formula (1). The crystalline formula (1) compound exhibits a CuKα X-ray diffraction spectrum having a diffraction peak at a Bragg angle 2θ (±0.2) in the range of 4.0° to 5.0°. The diffraction peak has a half-value width of 0.7° or more.

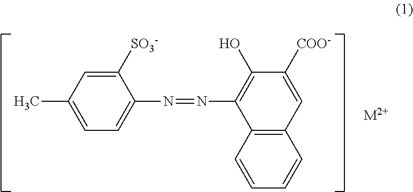

In formula (1), M represents an atom selected from the group consisting of barium, strontium, calcium, and manganese.

In the following description, the crystalline compound represented by formula (1) may be simply referred to as the formula (1) compound.

The crystalline formula (1) compound exhibits a CuKα X-ray diffraction spectrum having a diffraction peak having a half-value width of 0.7° or more at a Bragg angle 2θ (±0.2) in the range of 4.0° to 5.0°. Such a compound and the binder resin interact with each other and impart charging stability and a color stability to the toner.

Beneficially, in the CuKα X-ray diffraction spectrum of the crystalline formula (1) compound, the diffraction peak at a Bragg angle 2θ (±0.2) in the range of 4.0° to 5.0° has a half-value width in the range of 0.7° to 1.5°. The half-value width of the diffraction peak may be in the range of 0.8° to 1.2°. If the diffraction peak has a half-value width of less than 0.7°, the crystals of compound (1) are in a largely grown state. In such a case, the formula (1) compound does not interact easily with the binder resin and, accordingly, does not impart satisfactorily charging stability to the toner. In contract, if the half-value width of the diffraction peak is excessively large, the crystals are likely to be in a state insufficient in crystal growth. In such a case, the formula (1) compound is unlikely to exhibit a satisfactory color developability (tinting strength).

The proportion of the formula (1) compound in the toner relative to 100 parts by mass of the binder may be in the range of 1.0 part by mass to 20.0 parts by mass, beneficially in the range of 3.0 parts by mass to 20.0 parts by mass. More beneficially, it is in the range of 5.0 parts by mass to 15.0 parts by mass. If the proportion of the formula (1) compound is excessively low, a large amount of toner is required to output an image with a desired density. In contrast, if the proportion of the formula (1) compound is excessively high, the pigment particles are likely to aggregate in the toner particles, reducing the charging stability of the toner.

The toner particle of the toner of the present disclosure may further contain a quinacridone pigment (pigment dominantly containing a quinacridone-based compound). The quinacridone pigment further improves the charging stability and color stability of the toner. The proportion of the quinacridone pigment may be in the range of 4.0 parts by mass to 10.0 parts by mass relative to 100 parts by mass of the binding resin. When the quinacridone pigment is contained with such a proportion, the charging stability and color stability of the toner is further improved.

### Binder Resin

Examples of the binder resin contained in the toner particle include:
homopolymers of substituted or unsubstituted styrene, such as polystyrene, poly-p-chlorostyrene, and polyvinyl toluene;
styrene-based copolymers, such as styrene-p-chlorostyrene copolymer, styrene-vinyl toluene copolymer, styrene-vinyl naphthalene copolymer, styrene-acrylic acid ester copolymer, styrene-methacrylic acid ester copolymer, styrene-methyl α-chloromethacrylate copolymer, styrene-acrylonitrile copolymer, styrene-vinyl methyl ether copolymer, styrene-vinyl ethyl ether copolymer, styrene-vinyl methyl ketone copolymer, and styrene-acrylonitrile-indene copolymer;
polyvinyl chloride resin;
phenolic resin;
natural-modified phenolic resin;
natural-modified maleic acid resin;
acrylic resin;
methacrylic resin;
polyvinyl acetate resin;
silicone resin.;
polyester resin;
polyurethane resin;
polyamide resin;
furan resin;
epoxy resin;
xylene resin;
polyvinyl butyral resin;
terpene resin;
coumarone-indene resin; and
petroleum-based resin.

Among these, polyester resin is beneficially in view of charging stability.

The polyester resin used herein refers to resins having a polyester unit in the molecular chain thereof. The polyester unit may be made up of a divalent or higher valent alcohol monomer and a divalent or higher valent acid monomer, such as a divalent or higher valent carboxylic acid, a divalent or higher valent carboxylic anhydride, or a divalent or higher valent carboxylic acid ester.

Examples of the divalent or higher valent alcohol monomer include alkylene oxide adducts of bisphenol A, such as polyoxypropylene (2.2) -2,2-bis (4-hydroxyphenyl) propane, polyoxypropylene (3.3)-2,2-bis(4-hydroxyphenyl)propane, polyoxyethylene (2.0)-2,2-bis(4-hydroxyphenyl)propane, polyoxypropylene (2.0)-polyoxyethylene (2.0)-2,2-bis(4-hydroxyphenyl)propane, and polyoxypropylene (6)-2,2-bis(4-hydroxyphenyl)propane; and ethylene glycol, diethylene glycol, triethylene glycol, 1,2-propylene glycol, 1,3-propylene glycol, 1,4-butanediol, neopentyl glycol, 1,4-butenediol, 1,5-pentanediol, 1,6-hexanediol, 1,4-cyclohexanedimethanol, dipropylene glycol, polyethylene glycol, polypropylene glycol, polytetramethylene glycol, sorbitol, 1,2,3,6-hexanetetrol, 1,4-sorbitan, pentaerythritol, dipentaerythritol, tripentaerythritol, 1,2,4-butanetriol, 1,2,5-pentanetriol, glycerin, 2-methylpropanetriol, 2-methyl-1,2,4-butanetriol, trimethylolethane, trimethylolpropane, and 1,3,5-trihydroxymethylbenzene.

Aromatic diols are beneficially used and may account for 80% by mole or more of all the alcohol monomers in the polyester resin.

Exemplary divalent or higher valent acid monomers include:
aromatic dicarboxylic acids, such as phthalic acid, isophthalic acid, and terephthalic acid, and anhydrides thereof;
alkyl dicarboxylic acids, such as succinic acid, adipic acid, sebacic acid, and azelaic acid, and anhydrides thereof;
succinic acids substituted by an alkyl or alkenyl group having a carbon number of 6 to 18 and anhydrides thereof; and
unsaturated dicarboxylic acids, such as fumaric acid, maleic acid, and citraconic acid, and anhydrides thereof.

Among these beneficial are multivalent carboxylic acids, such as terephthalic acid, succinic acid, adipic acid, fumaric acid, trimellitic acid, pyromellitic acid, and benzophenone tetracarboxylic acid, and anhydrides thereof.

The polyester resin may have an acid value in the range of 0 mg K /g to 20 mg KOH/g in view of dispersibility of the pigment and stability in development. Beneficially, it is in the range of 0 mg KOH/g to 15 mg KO/g.

If the acid value of the polyester resin is excessively high, the formula (1) compound is unlikely to be satisfactorily dispersed in the toner particle, degrading the charging stability of the toner.

The acid value of the polyester resin can be controlled by varying the monomers used for synthesizing the polyester resin and the amount thereof. For example, the acid value may be controlled by adjusting the proportion of the alcohol monomer to the acid monomer in synthesis of the polyester and the molecular weights of these monomers. Alternatively, after a condensation polymerization for esterification, the terminal alcohol may be allowed to react with a multivalent acid monomer (such as trimellitic acid). Resin Having Structure Formed by Reaction Between Vinyl-Based Resin Component and Hydrocarbon

In an embodiment, the toner particle may contain a resin having a structure formed by a reaction between a vinyl-based resin component and a hydrocarbon compound. Such a resin helps the formula (1) compound to disperse finely and uniformly in the toner particle.

The resin having a structure formed by a reaction between a vinyl-based resin component and a hydrocarbon compound may be a graft copolymer having a structure in which a polyolefin is grafted onto a vinyl-based resin component, or a graft copolymer containing a vinyl-based resin component formed by grafting a vinyl-based monomer onto a polyolefin.

The resin having a structure formed by a reaction between a vinyl-based resin component and a hydrocarbon compound acts like a surfactant for the binder resin and wax in the steps of kneading and surface smoothing in the manufacture of the toner. Thus, this resin helps adjust the average primary particle size of the wax dispersed in the toner particle, and helps adjust the speed of the wax moving to the surfaces of the toner particles when, if necessary, the toner particles are surface treated with hot air.

For synthesis of the graft copolymer having a structure in which a polyolefin is grafted onto a vinyl-based resin monomer or the graft copolymer containing a vinyl-based resin component formed by grafting a vinyl-based monomer onto a polyolefin, the polyolefin may be selected from various polyolefins. The polyolefin may be a homopolymer or copolymer of one or more unsaturated hydrocarbon monomers having a single double bond. Polyethylene-based or polypropylene-based polyolefins are beneficial as the polyolefin.

Examples of the vinyl monomer include:
styrene-based monomers, such as styrene, o-methylstyrene, m-methylstyrene, p-methylstyrene, p-methoxystyrene, p-phenylstyrene, p-chlorostyrene, 3,4-dichlorostyrene, p-ethylstyrene, 2,4-dimethylstyrene, p-n-butylstyrene, p-tert-butylstyrene, p-n-hexyistyrene, p-n-octylstyrene, p-n-nonylstyrene, p-n-decylstyrene, p-n-dodecylstyrene, and derivatives thereof;
amino-containing α-methylene aliphatic monocarboxylic acid esters, such as dimethylaminoethyl methacrylate and diethylaminoethyl methacrylate;
nitrogen-containing vinyl monomers, such as acrylonitrile, methacrylonitrile, acrylamide, and other acrylic acid or methacrylic acid derivatives;
unsaturated dibasic acids, such as maleic acid, citraconic acid, itaconic acid, alkenylsuccinic acids, fumaric acid, and mesaconic acid;
unsaturated dibasic acid anhydrides, such as maleic anhydride, citraconic anhydride, itaconic anhydride, and alkenylsuccinic anhydrides;
half esters of unsaturated dibasic acids, such as maleic acid methyl half ester, maleic acid ethyl half ester, maleic acid butyl half ester, citraconic acid methyl half ester, citraconic acid ethyl half ester, citraconic acid butyl half ester, itaconic acid methyl half ester, alkenylsuccinic acid methyl half ester, fumaric acid methyl half ester, and mesaconic acid methyl half ester;
unsaturated dibasic acid esters, such as dimethyl maleate and dimethyl fumarate;
αβ-unsaturated acids, such as acrylic acid, methacrylic acid, crotonic acid, and cinnamic acid;
αβ-unsaturated acid anhydrides, such as crotonic anhydride and cinnamic anhydride, and anhydrides of any of the αβ-unsaturated acids with a lower fatty acid;
carboxy-containing vinyl monomers, such as alkenylmalonic acids, alkenylglutaric acids, alkenyladipic acids, anhydrides thereof, and monoesters thereof;
hydroxy-containing vinyl monomers, such as 2-hydroxyethyl acrylate, 2-hydroxyethyl methacrylate, 2-hydroxypropyl methacrylate, similar acrylic or methacrylic acid esters, (1-hydroxy-1-methylbutyl)styrene, and 4-(1-hydroxy-1-methylhexyl)styrene;
other acrylic acid esters, such as methyl acrylate, ethyl acrylate, n-butyl acrylate, isobutyl acrylate, propyl acrylate, n-octyl acrylate, dodecyl acrylate, 2-ethylhexyl acrylate, stearyl acrylate, 2-chloroethyl acrylate, and phenyl acrylate; and
other methacrylic acid esters, such as methyl methacrylate, ethyl methacrylate, n-butyl methacrylate, isobutyl methacrylate, propyl methacrylate, n-octyl methacrylate, dodecyl methacrylate, 2-ethylhexyl methacrylate, stearyl methacrylate, phenyl methacrylate, and dimethylaminoethyl methacrylate and other α-methylene aliphatic monocarboxylic acid esters.

The resin having a structure formed by a reaction between a vinyl-based resin component and a hydrocarbon compound may be produced by a reaction of any two or more of the above-cited monomers or a reaction between one of the monomers of the polymer and the other.

The vinyl-based resin component may contain a unit derived from a styrene-based monomer and, in addition, a unit derived from acrylonitrile and/or methacrylonitrile.

In this resin, the mass ratio of the hydrocarbon compound to the vinyl-based resin component (hydrocarbon/vinyl-based resin component ratio) may be in the range of 1/99 to 75/25 from the viewpoint of satisfactorily dispersing the pigment in the toner particle.

In the toner particle, the proportion of the resin having a structure formed by a reaction between a vinyl-based resin component and a hydrocarbon compound may be in the range of 0.2 part by mass to 20 parts by mass relative to 100 parts by mass of the binder resin. Beneficially, it is in the range of 3.0 parts by mass to 10 parts by mass.

The resin having a structure formed by a reaction between a vinyl-based resin component and a hydrocarbon compound may have a weight average molecular weight (Mw) in the range of 6000 to 8000 from the viewpoint of satisfactory dispersing the pigment in the toner particle. The number average molecular weight (Mn) may be in the range of 1500 to 5000 from the same viewpoint.

### Wax

In an embodiment, the toner particle may optionally contain a wax.

Examples of the wax include:
hydrocarbon waxes, such as low-molecular-weight polyethylene, low-molecular-weight polypropylene, alkylene copolymers, microcrystalline waxes, paraffin waxes, and Fischer-Tropsch waxes;
oxidized hydrocarbon waxes, such as oxidized polyethylene waxes, and block copolymers thereof;
fatty acid ester-based waxes, such as carnauba wax; fully or partially deoxidized fatty acid esters, such as deoxidized carnauba wax. The following compounds may be used as the wax: saturated straight-chain fatty acids, such as palmatic acid, stearic acid, and montanic acid;
unsaturated fatty acids, such as brassidic acid, eleostearic acid, and parinaric acid;
saturated alcohols, such as stearyl alcohol, aralkyl alcohol, behenyl alcohol, carnaubyl alcohol, ceryl alcohol, and melissyl alcohol;
polyhydric alcohols, such as sorbitol;
esters of a fatty acid such as palmitic acid, stearic acid, behenic acid, or montanic acid with an alcohol such as stearyl alcohol, aralkyl alcohol, behenyl alcohol, carnaubyl alcohol, ceryl alcohol, or melissyl alcohol;
fatty acid amides, such as linoleic acid amide, oleic acid amide, and lauric acid amide;
saturated fatty acid bisamides, such as methylene-bis-stearic acid amide, ethylene-bis-capric acid amide, ethylene-bis-lauric acid amide, and hexamethylene-bis-stearic acid;
unsaturated fatty acid amides, such as ethylene-bis-oleic acid amide, hexamethylene-bis-oleic acid amide, N,N′-dioleyladipic acid amide, and N,N′-dioleylsebacic acid amide;
aromatic bisamides, such as m-xylene bis-stearic acid amide and N,N′-distearylisophthalic acid amide;
fatty acid metal salts (generally referred to as metallic soap), such as calcium stearate, calcium laurate, zinc stearate, and magnesium stearate;
aliphatic hydrocarbon waxes grafted with a vinyl-based monomer, such as styrene or acrylic acid;
partial esterification products of a fatty acid with a polyhydric alcohol, such as behenic acid monoglyceride; and hydroxy-containing methyl ester compounds produced by hydrogenation of vegetable oil or fat.

From the viewpoint of improving low-temperature fixability, hot-offset resistance, and resistance to winding around the fixing device, paraffin waxes and Fischer-Tropsch waxes are beneficial.

The proportion of the wax in the toner particle may be in the range of 0.5 part by mass to 20 parts by mass relative to 100 parts by mass of the binder resin. Beneficially, it is in the range of 3.0 parts by mass to 12 parts by mass.

Beneficially, the endothermic curve of the wax measured during heating with a differential scanning calorimeter (DSC) has a peak (derived from the wax) in the range of 30° C. to 200° C., and the highest temperature of the peak is in the range of 50° C. to 110° C., from the viewpoint of achieving a toner having both good storage stability and high hot-offset resistance. More beneficially, the highest endothermic peak temperature is in the range of 70° C. to 100° C.

### Charge Control Agent

In an embodiment, the toner particle may optionally contain a charge control agent.

The charge control agent may be a colorless aromatic carboxylic acid metal compound that enables the toner to be rapidly charged and stably holds a constant amount of charge.

Examples of such a negative charge control agent include:
salicylic acid metal compounds,
naphthoic acid metal compounds,
dicarboxylic acid metal compounds,
polymers having a side chain having sulfonic acid or a carboxylic acid,
polymers having a side chain having sulfonic acid or an ester thereof,
polymers having a side chain having a carboxylic acid or an ester thereof,
boron compounds,
urea compounds,
silicon compounds, and
calixarene.

The charge control agent may be added into each toner particle or externally added to the mass of the toner particles.

The proportion of the charge control agent in the toner may be in the range of 0.2 part by mass to 10 parts by mass relative to 100 parts by mass of the binder resin.

### External Additive

In an embodiment, an external additive may optionally be added (externally added) to the mass of the toner particles from the viewpoint of improving the fluidity of the toner and controlling the triboelectric charge on the toner.

The external additive may be fine particles of an inorganic compound, such as silica (silicon dioxide), titanium oxide, aluminum oxide, or strontium titanate.

The inorganic fine particles may be hydrophobized with a hydrophobizing agent, such as a silane compound, silicone oil, or a mixture thereof.

From the viewpoint of preventing the external additive from sinking in the mass of the toner particles, the external additive may have a specific surface area in the range of 10 m²/g to 50 m²/g.

The proportion of the external additive may be in the range of 0.1 part by mass to 5.0 parts by mass relative to 100 parts by mass of the toner particles.

For mixing the toner particles with the external additive, a mixer such as a Henschel mixer may be used.

The toner of an embodiment of the present disclosure may be mixed with a magnetic carrier for use as a two-component developer.

Examples of the magnetic carrier include surface-oxidized or unoxidized iron powder; particles of metal, such as lithium, calcium, magnesium, nickel, copper, zinc, cobalt, manganese, and rare-earth metals, and alloy particles or oxide particles thereof; ferrite and similar magnetic substances; and magnetic substance-dispersed resin carriers (what are called resin carrier) containing a magnetic substance and a binder resin capable of keeping the magnetic substance dispersed.

### Manufacturing Method

Various processes may be applied to the manufacture of the toner of the present disclosure.

A method using pulverization will now be described for manufacturing the toner.

In the step of mixing ingredients, ingredients of the toner particles including a binder resin and a wax and optional ingredients, such as a coloring agent and a charge control agent are mixed with predetermined proportions. Examples of the mixer used in this step include double-cone mixers, V-shaped mixers, drum mixers, super mixers, Henschel mixers, Nauta mixers, and Mechano Hybrid manufactured by Nippon Coke & Engineering.

Subsequently, the mixture is melt-kneaded to disperse the wax and optional ingredients in the binder resin. For the melt-kneading, a kneader, such as a pressure kneader, a Banbury mixer or any other batch-type kneading device, or a continuous kneading device, may be used. From the viewpoint of continuous production, a single-screw or twin-screw extruder may be used. Such a kneader or truder may be, for example, a twin-screw extruder model KTK (manufactured by Kobe Steel) or a twin-screw extruder model TEM (manufactured by Toshiba Machine). Other examples Include PCM kneader manufactured by Ikegai, a twin-screw extruder manufactured by KCK, a co-kneader manufactured by Buss, and Kneadex manufactured by Nippon Coke & Engineering. The resin composition prepared by melt-kneading may be rolled with a two-roll mill or the like, and cooled with water in a cooling step.

The resin composition or the cooled resin composition is pulverized into particles having a desired particle size. In pulverization, the resin composition is roughly crushed with a crusher and then further pulverized into fine particles with a pulverizer. The rough crushing may be performed with, for example, a crusher, a hammer mill, a feather mill, or the like. For the subsequent pulverization, a pulverization apparatus may be used, such as a Kryptron system (manufactured by Kawasaki Heavy industries), Super Roater (manufactured by Nisshin Engineering), a turbo mill (manufactured by Freund Turbo), or an air-Jet pulverizer.

The resulting fine particles are optionally sized with a classifier or a sifter to yield toner particles. The classifier or sifter may be an inertial classification classifier Elbow-Jet (Nittetsu Mining), a centrifugal classifier Turboplex (manufactured by Hosokawa Micron), TSP Separator (manufactured by Hosokawa Micron), or Faculty (manufactured by Hosokwawa Micron).

Then, an external additive, such as inorganic particles or resin particles, may optionally be added to (mixed with) the mass of the toner particles to impart a fluidity to the particles or increase the charging stability of the toner. Thus, the toner is produced. For mixing the external additive, a mixer including a rotation device having a stirring member, and a body casing disposed with a gap from the stirring member may be used.

Examples of such a mixer include Henschel Mixer (manufactured by Nippon Coke & Engineering), Super Mixer (manufactured by Kawata), Ribocone (manufactured by Okawara MFG.), and Nauta Mixer, Turbulizer, and Cyclomix (each manufactured by Hosokawa Micron). Also, Spiral Pin Mixer (manufactured by Pacific Machinery & Engineering), Loedige Mixer (manufactured by Matsubo), or Nobilta (manufactured by Hosokawas Micron) may be used. From the viewpoint of uniformly mixing the toner and the external additive and disentangling aggregates of the external additive such as silica particles, Henschel mixer may be beneficially used.

The conditions to be controlled for the mixing include the amount of toner, the rotational speed of the stirring shaft, the stirring time, the shape of stirring blade, the temperature in the casing or stirring chamber, or the like.

Then, if large aggregates of the external additive remain in the resulting toner, the toner may be shifted, if necessary.

Physical properties of the toner and the ingredients of the toner may be measured as follows. Measurement of Peak Molecular Weight (Mp), Number Average Molecular Weight (Mn), and Weight Average Molecular Weight (Mw) of the Resin

The peak molecular weight (Mp), the number average molecular weight (Mn), and the weight average molecular weight (Mw) may be measured by gel permeation chromatography (GPC) as below.

First, a sample (resin) is dissolved in tetrahydrofuran (THF) at room temperature over a period of 24 hours. The resulting solution is filtered through a solvent-resistant membrane filter “Maeshori Disk” of 0.2 μm in pore size (manufacture by Tosoh Corporation) to yield a sample solution. The sample solution is adjusted so that the content of the constituent soluble in THF will be about 0.8% by mass. The resulting sample solution is subjected to measurement under the following conditions:
Apparatus: HLC 8120 GPC (detector: RI) (manufactured by Tosoh Corporation)
Columns: combination of 7 columns of Shodex series KF-801, KF-802, KF-803, KF-804, KF-805, KF-806, and KF-807 (manufactured by Showa Denko)
Fluent: tetrahydrofuran (THF)
Flow rate: 1.0 mL/min
Oven temperature: 40.0° C.
Volume of sample injected: 0.10 mL

For calculating the molecular weight of the sample, a molecular weight calibration curve is prepared by using standard polystyrene resins. Exemplary standard polystyrene resins include TSK Standard Polystyrenes F-850, F-450, F-288, F-128, F-80, F-40, F-20, F-10, F-4, F-2, F-1, A-5000, A-2500, A-1000, and A-500 (each produced by Tosoh).

### Measurement of Softening Point of the Resin

The softening point of a resin sample is measured with a capillary rheometer of a constant-pressure extrusion system using toad, Flow Tester CFT-500D (manufactured by Shimadzu), in accordance with the manual attached to the tester. In this apparatus, the measurement sample in a cylinder is heated to be melted while a constant load is placed on the measurement sample by a piston, and the melted sample is extruded from the cylinder. Thus, by using this apparatus, a rheogram showing the relationship between the downward displacement of the piston and the heating temperature at this time can be prepared. The softening point mentioned herein is defined as the melting temperature measured by the 1/2 method described in the manual attached to the flow tester CFT-500D. The melting temperature determined by the 1/2 method is obtained as below. First calculated is a half X of the difference between the downward displacement Smax of the piston at the time when the sample has flowed out completely and the downward displacement Smin of the piston at the time when the sample has started flowing. X=(Smax−Smin)/2 The temperature in the rheogram at which the downward displacement of the piston comes to X is the melting temperature measured by the 1/2 method.

For this measurement, about 1.0 g of a resin sample is compacted into a cylindrical tablet with a diameter of about 8 mm in a tablet forming machine (for example, NT-100H manufactured by NPa System) at about 10 MPa over a period of about 60 seconds under an environment of 25° C. This tabled is used as the measuring sample.

The measurement using CFT-500D is performed under the following conditions:
Test mode: heating
Start temperature: 40° C.
End-point temperature: 200° C.
Measuring temperatures: 1.0° C. increments
Heating rate: 4.0° C./min
Cross section of piston: 1.000 cm²
Testing load (piston load) : 10.0 kgf (0.9807 MPa)
Preheating time: 300 s
Hole diameter in die: 1.0 mm
Die length: 1.0 mm

### Measurement of Acid Value

The acid value of a sample (resin) refers to the mass (milligrams) of potassium hydroxide required to neutralize the acid contained in 1 g of the sample. The acid value is measured in accordance with JIS K 0070-1992, specifically as below.

### (1) Preparation of Regents

A phenolphthalein solution is prepared by dissolving 1.0 g of phenolphthalein in 90 mL of 95% by volume ethyl alcohol and adding ion-exchanged water up to a total volume of 100 mL.

In 5 mL of water is dissolved 7 g of highest-quality potassium hydroxide, and ethyl alcohol (95% by volume) is added up to a total volume of 1 L. The mixture is allowed to stand for 3 days in an alkali-resistant container so as not to come into contact with carbon dioxide or the like. Then, the mixture is filtered to yield a potassium hydroxide solution. The resulting potassium hydroxide solution is stored in an alkali-resistant container. The factor of the potassium hydroxide solution is determined from the amount of the potassium hydroxide solution used for titration for neutralizing 25 mL of 0.1 mol/L hydrochloric acid solution in a conical flask to which some droplets of the phenolphthalein solution have been added. The 0.1 mol/L hydrochloric acid solution is prepared in accordance with JIS K 8001-1998.

### (2) Operation

### (A) Main Test

To accurately weighed 2.0 g of a resin sample in a 200 mL conical flask is added 100 mL of toluene/ethanol (2:1) mixed solution, and the sample is dissolved over a period of 5 hours. Subsequently, some droplets of the phenolphthalein solution are added as an indicator, and the resulting resin solution is titrated with the above-prepared potassium hydroxide solution. The end point of the titration is when the indicator turns pink and the pink color is kept for 30 seconds.

### (B) Blank Test

The same operation as above is performed without using the resin sample (only toluene/ethanol (2:1) mixed solution is titrated).

### (3) Calculation

The acid value of the resin sample is calculated by using the titration result and the following equation:

A=[(C−B)×f×5.61]/S
wherein A represents the acid value (mg KOH/a) of the sample; B represents the volume (mL) of the potassium hydroxide solution added in the blank test; C represents the volume (mL) of the potassium hydroxide solution added in the main test; f represents the factor of the potassium hydroxide solution; and S represents the weight (g) of the sample.

### Measurement of Hydroxy Value

The hydroxy value refers to the mass (milligrams) of potassium hydroxide required to neutralize the acetic acid bound to hydroxy groups for acetylation of 1 g of a sample. The hydroxy value of a resin is measured in accordance with JIS K 0070-1992, specifically as below.

### (1) Preparation of Regents

Pyridine is added into a 100 mL measuring flask containing 25 g of highest-quality acetic anhydride up to a total volume of 100 mL. The mixture is sufficiently shaken to yield an acetylation reagent. The acetylation reagent is stored in a brown bottle so as not to come into contact with moisture, carbon dioxide, and the like.

A phenolphthalein solution is prepared by dissolving 1.0 g of phenolphthalein in 90 mL of 95% by volume ethyl alcohol and adding ion-exchanged water up to a total volume of 100 mL.

In 20 mL of water is dissolved 35g of highest-quality potassium hydroxide, and ethyl alcohol (95% by volume) is added up to a total volume of 1 L. The mixture is allowed to stand for 3 days in an alkali-resistant container so as not to come into contact with carbon dioxide or the like. Then, the mixture is filtered to yield a potassium hydroxide solution. The resulting potassium hydroxide solution is stored in an alkali-resistant container. The factor of the potassium hydroxide solution is determined from the amount of the potassium hydroxide solution used for titration for neutralizing 25 mL of 0.5 mol/L hydrochloric acid solution in a conical flask to which some droplets of the phenolphthalein solution have been added. The 0.5 mol/L hydrochloric acid solution is prepared in accordance with JIS K 8001-1998.

### (2) Operation

### (A) Main Test

Accurately weighed 1.0 g of crushed resin sample is placed in a 200 mL round-bottom flask, and exactly 5.0 mL of the acetylation reagent is added with a whole pipette. If the sample is difficult to dissolve in the acetylation reagent, a small amount of highest-quality toluene is added to help the reagent to dissolve.

The flask with a small funnel on the top thereof is heated in a glycerin bath of about 97° C. in such a manner that the portion of the flask 1 cm from the bottom is immersed in the glycerin. At this time, the flask may be provided with a paperboard or the like with a round hole therein in such a manner that the neck of the flask passes through the hole, thus preventing the neck from being heated by the heat of the bath.

After 1 hour, the flask was removed from the glycerin bath and allowed to cool down. After cooling down, 1 mL of water is added into the flask through the funnel, and the flask is shaken for hydrolysis of the acetic anhydride. For complete hydrolysis, the flask is further heated in the glycerin bath for 10 minutes. After allowing the flask to cool down, the walls of the funnel and flask are rinsed with 5 mL of ethyl alcohol.

Some droplets of the phenolphthalein solution are added as an indicator into the flask, and the solution in the flask is titrated with the above-prepared potassium hydroxide solution. The end point of the titration is when the indicator turns pink and the pink color is kept for 30 seconds.

### (B) Blank Test

The same operation as above is performed except that the resin sample is not used.

### (3) Calculation

The hydroxy value of the resin sample is calculated by using the titration result and the following equation:

A=[{(B−C)×28.05×f}/S]+D
wherein A represents the hydroxy value (mg KOH/g) of the sample; B represents the volume (mL) of the potassium hydroxide solution added in the blank test; C represents the volume (mL) of the potassium hydroxide solution added in the main test; f represents the factor of the potassium hydroxide solution; and S represents the weight (g) of the sample. D represents the acid value (mg KOH/g) of the resin sample.

### Measurement of Highest Endothermic Peak Temperature of Wax

The highest endothermic peak temperature of the wax is measured according to ASTM D3418-82 with a differential scanning calorimeter Q1000 (manufacture by TA Instruments). For the temperature compensation of the detector of the calorimeter, the melting points of indium and zinc are used. The amount of heat is corrected using the heat of fusion of indium.

More specifically, about 10 mg of wax is placed in an aluminum pan and measured at a temperature in the range of 30° C. to 200° C. at a heating rate of 10° C/min, using an empty aluminum pan as a reference. In this measurement, the sample is heated to 200° C. once, subsequently cooled to 30° C., and then heated again. The temperature at which the endothermic peak of the DSC curve in the temperature range of 30° C. to 200° C. measured in the second heating becomes highest is defined as the highest endothermic peak temperature of the wax.

### X-Rav Diffraction of Toner

For X-ray diffraction, an instrument RINT-TTR II (manufactured by Rigaku) and a control and an analysis software program attached to the instrument are used.

The measurement is performed under the following conditions:
X-ray radiation: Cu/50 kV/300 mA
Goniometer: Rotor horizontal goniometer (TTR-2)
Attachment: Standard sample holder
Divergence slit: open
Divergence vertical limit slit: 10.00 mm
Scattering slit: open
Receiving slit: open
Counter: scintillation counter
Scanning mode: continuous
Scanning speed: 4.0000° /min
Sampling width: 0.0200°
Scanning axis: 2θ/θ
Scanning range: 10.0000° to 40.0000°

Subsequently, a powdery sample is measured on a sample plate. The sample is subjected to CuKα X-ray diffraction at Bragg angles (2θ±0.2°) in the range of 3.0° to 35.0°, and the half-value width in the obtained spectrum in the 2θ range of 4.0° to 5.0° is defined as an indicator of the crystallinity (degree of crystal growth).

X-Ray Diffraction of Formula (1) Compound Isolated from Toner

When the formula (1) compound isolated from a toner is subjected to X-ray diffraction, the toner is dissolved in tetrahydrofuran (THF) or chloroform. The undissolved phase and the dissolved phase are separated from each other with a Soxhlet extractor. The undissolved phase is sufficiently dried and allowed to stand under the conditions of 23° C. and 60% RH for 24 hours or more to yield a measurement sample. The isolated sample, or formula (1) compound, is subjected to X-Ray diffraction under the same conditions as in the case of toner.

### Measurement of Spectral Reflectance of Toner

An aluminum ring of 30 mm in diameter is charged with 3 g of toner, and the toner is formed into a pellet at a pressure of 10 t. The reflectance of the toner is measured with a spectroscopic color difference meter SE-2000 (manufactured by Nippon Denshoku Industries). The reflectance in the range of 400 nm to 500 nm and in the range of 650 nm to 700 nm is measured in 10 nm increments, and the average is calculated. The spectral reflectance of the toner may be controlled by selecting the compound or pigment in the toner.

Beneficially, the reflectance of the toner for a wavelength in the range of 400 nm to 500 nm is 25% or less. Also, the reflectance of the toner for a wavelength in the range of 650 nm to 700 nm is, beneficially, 90% or more.

### EXAMPLES

The subject matter of the disclosure will be further described in detail with reference to Examples below. In the following description, the term “part(s)” refers to “part(s) by mass”.

### Preparation of Formula (1) Compound

4-Aminotoluene-3-sulfonic acid: 200.0 parts
4-Amino-2-chlorotoluene-5-sulfonic acid: 20.0 parts
2-Aminonaphthalene-5-sulfonic acid: 2.0 parts

These ingredients were dispersed in 1000 parts of water, and 226.4 parts of 20% hydrochloric acid was added to the dispersion. Then, 190 parts of 40% sodium nitrite aqueous solution was dropped into the dispersion whose temperature is kept at 0° C. by adding ice thereto. Then, water was added up to a total of 4000 parts to yield a suspension of a diazonium salt.

Next, 210.2 parts of 2-hydroxy-3--naphthoic acid was dispersed in 1600 parts of hot water of 60° C., and then 400 parts of 25% sodium hydroxide aqueous solution was added. Subsequently, water was added up to a total of 5000 parts to yield a coupler solution.

While the coupler solution cooled to 0° C. or less by adding cold water was being stirred, the suspension of the diazonium salt was added to the coupler solution at a rate of 62 parts per minute.

After the entirety of the suspension of the diazonium sale was added, the mixture was stirred at temperature of 0° C. to 3° C. for 60 minutes. To the resulting suspension was added 545.6 parts of a solution of disproportionated rosin potassium salt, followed by stirring for 30 minutes. After adding sodium hydroxide to adjust the pH, an aqueous solution containing 241.3 parts of calcium chloride (purity: 75%) was added for laking (conversion into a lake), followed by stirring for 60 minutes.

Then, the contents of the reaction system were stirred while being heated at 85° C. for 60 minutes, filtered, rinsed, and dried to yield powder of a formula (1) compound.

The formula (1) compound exhibited a CuKα X-ray diffraction spectrum in which the diffraction peak at a Bragg angle 2θ (±0.2) in the range of 4.0° to 5.0° had a half-value width of 0.9°. The half-value width can be controlled by changing the metal salt used for laking in the process for producing the formula (1) compound and the heating temperature after the laking.

### Preparation of Coloring Agent 1

ion exchanged water: 1500 parts
Formula (1) Compound: 100.0 parts

These ingredients were mixed with stirring to suspend the formula (1) compound in water. Then, 15.0 parts of tetrahydroabietic acid, 5.0 pats of abietic acid, and 30 parts of 33% sodium hydroxide aqueous solution were added to the suspension. The mixture was heated to 98° C. and then stirred for 1 hour while being kept at this temperature. After cooling to 65° C., about 60 parts of 31% hydrochloric acid was added to precipitate a resin. The precipitate was separated out by filtration, rinsed with ion-exchanged water, and dried to yield coloring agent 1.

Coloring agent 1 exhibited a CuKα X-ray diffraction spectrum in which the diffraction peak at a Bragg angle 20 (±0.2) in the range of 4.0° to 5.0° had a half-value width of 0.9°.

### Preparation of Coloring Agents 2- to 7

Coloring agents 2 to 7 were prepared in the same manner as in the process of coloring agent 1, except that the compound shown in Table 1 was prepared as the formula (1) compound.

Coloring agents 2 to 7 each exhibited a CuKα X-ray diffraction spectrum in which the diffraction peak at a Bragg angle 2θ (±0.2) in the range of 4.0° to 5.0° had a half-value width shown in Table 1.

### Preparation of Coloring Agents 8 to 1.0

Into a pressure reactor or autoclave were added 30.00 parts of dimethyl succinylosuccinate (1,4-cyclohexanedione-2,5-di-carboxylic acid methyl ester, 7.00 parts of aniline, 22.00 parts of toluidine, 300.00 parts of methanol, and 1.00 part of 35% by mass hydrochloric acid to prepare a mixture.

The autoclave was sealed and purged with nitrogen, and the interior of the autoclave was holed at a gauge pressure of 0.1 kg/cm². The interior of the autoclave was heated from 25° C. to 85° C. at a rate of 4.0° C./min while the mixture was stirred, and the mixture was subjected to a reaction at 85° C. for 5 hours to yield a reaction mixture.

Then, when the reaction mixture had been cooled to 30° C. or less, the pressure was released to atmospheric pressure. The cooling was continued, and the interior of the autoclave was kept at 25° C.

Into the autoclave were added 40.00 parts of 50% by mass sodium hydroxide aqueous solution and 34.60 parts of sodium m-nitrobenzenesulfonate to yield mixture 2, and the autoclave was sealed.

The mixture 2 was stirred for 10 minutes, and the interior of the autoclave was heated from 25° C. to 85° C. at a rate of 4.0° C./min for a reaction of mixture 2 for 5 hours. Then, the interior of the autoclave was cooled to 30° C. or less, and the contents of the autoclave were filtered to remove all the solids.

The remaining solution was heated to 40° C. while being stirred, and 18.00 parts of 35% by mass hydrochloric acid was dropped into the solution. The resulting mixture was kept at this temperature for 30 minutes.

Then, the mixture was filtered. The cake remaining after the filtration was rinsed with water/methanol mixture (volume ratio, 1/1) and cold water and then dried to yield a product.

Then, a stirring vessel was charged with 250.00 parts of polyphosphoric acid containing 85.0% by mass of P₂O₅, and the vessel was heated to 90° C. with stirring and kept at this temperature.

Into this stirring vessel was added 45 parts of the reaction product, followed by heating at 130° C. for 3 hours for a ring-closing reaction. The ring-closing reaction product was cooled to 110° C., and 6 parts of water was gradually added to the product over a period of 10 minutes.

Then, the ring-closing reaction product was poured into 750 parts of water of 50° C., and the mixture was stirred at 60° C. for 1.5 hours. The solids were collected by filtration and rinsed until rinsing water becomes neutral, thus yielding a pre-cake.

The pre-cake (100 parts) was slurried in 170 parts of methanol, and the resulting slurry was heated at 90° C. for 3 hours in a pressure-resistant reactor. The slurry was then cooled and adjusted to a pH in the range of 9.0 to 9.5 with 50% by mass sodium hydroxide solution.

The solids were collected by filtration and rinsed with water. The resulting wet solids were dried at 80° C. in an oven to yield a coloring agent. In the process for preparing coloring agents 8 to 10, the heating temperature and heating time for slurrying were varied.

Coloring agents 8 to 10 exhibited a CuKα X-ray diffraction spectrum having no peaks at a Bragg angle 2θ (±0.2) in the range of 4.0° to 5.0°.

TABLE 1

| | | | Components of | | |
|---|---|---|---|---|---|
| | | | compound | | |
| | Coloring agent | Basic structure of component | M | R¹ | R² |
| Coloring agent 1 Coloring agent 2 Coloring agent 3 Coloring agent 4 Coloring agent 5 | Pigment dominantly containing formula (1) compound | 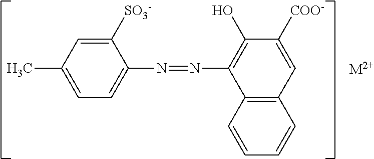 | Ca Ca Ca Mn Mn | — — — — — | — — — — — |
| Coloring | | | Ba | — | — |
| agent 6 | | | | | |
| Coloring | | | Ca | — | — |
| agent 7 | | | | | |
| Coloring agent 8 Coloring agent 9 Coloring agent 10 | Pigment dominantly containing quinacridone-based compound | 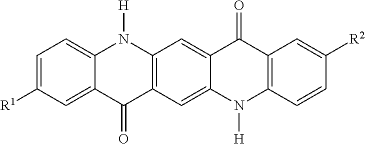 | — — — | CH₃ CH₃ H | CH₃ H H |

| | Heat treatment temperature (° C.) | Position (°) of | Half-value width (°) of |
|---|---|---|---|
| | after making lake | diffraction peak | diffraction peak |
| Coloring | 85 | 4.5 | 0.9 |
| agent 1 | | | |
| Coloring | 70 | 4.5 | 0.8 |
| agent 2 | | | |
| Coloring | 65 | 4.5 | 0.7 |
| agent 3 | | | |
| Coloring | 70 | 4.5 | 1.2 |
| agent 4 | | | |
| Coloring | 50 | 4.5 | 1.5 |
| agent 5 | | | |
| Coloring | 40 | 4.5 | 1.6 |
| agent 6 | | | |
| Coloring | 40 | 4.5 | 0.5 |
| agent 7 | | | |
| Coloring | — | — | — |
| agent 8 | | | |
| Coloring | — | — | — |
| agent 9 | | | |
| Coloring | — | — | — |
| agent 10 | | | |

### Preparation of Binder Resin 1

A 4 L four-neck glass flask was charged with the following ingredients:
76.9 parts (0.167 mol) of polyoxypropylene (2.2)-2,2-bis(4-hydroxyphenyl)propane;
24.1 parts (0.145 mol) of terephthalic acid;
8.0 parts (0.054 mol) of adipic acid; and
0.5 part of titanium tetrabutoxide.

The flask was placed in a heating mantle equipped with a thermometer, a stirrer, a condenser, and a nitrogen inlet.

After the flask was purge with nitrogen gas, the contents of the flask were gradually heated with stirring and allowed to react with stirring at 200° C. for 4 hours (first reaction).

Then, 1.2 parts (0.006 mol) of trimeflitic anhydride was added into the flask, and the contends of the flask were allowed to react at 180° C. for 1 hour (second reaction.) to yield binder resin. 1.

The acid value of binder resin 1 was 5 mg KOH/g, and the hydroxy value thereof was 65 mg KOH/g. Binder resin 1. was subjected to gel permeation chromatography (GPC) to measure the molecular weight. The weight average molecular weight (Mw) was 8,000; the number average molecular weight (Mn) was 3,500; and the peak molecular weight (Mp) was 5,700. Also, the softening point was 90° C.

### Preparation of Binder Resin 2

A 4 L four-neck glass flask was charged with the following ingredients:
71.3 parts (0.155 mol) of polyoxypropylene (2.2)-2,2-bis(4-hydroxyphenyl)propane;
24.1 parts (0.145 mol) of terephthalic acid; and
0.6 part of titanium tetrabutoxide.

The flask was placed in a heating mantle equipped with a thermometer, a stirrer, a condenser, and a nitrogen

After the flask was purge with nitrogen gas, the contents of the flask were gradually heated with stirring and allowed to react with stirring at 200° C. for 2 hours (first reaction).

Then, 5.8 parts (0.030 mol) of trimeflitic anhydride was added into the flask, and the contends of the flask were allowed to react at 180° C. for 10 hours (second reaction) to yield binder resin 2.

The acid value of binder resin 2 was 15 mg KOH/g, and the hydroxy value thereof was 7 mg KOH/g. Binder resin 2 was subjected to gel permeation chromatography (GPC) to measure the molecular weight. The weight average molecular weight (Mw) was 200,000; the number average molecular weight (Mn) was 5,000; and the peak molecular weight (Mp) was 10,000. Also, the softening point was 130° C.

### Preparation of Binder Resin 3

A 4 L four-neck glass flask was charged with the following ingredients:
76.9 parts (0.167 mol) of polyoxypropylene (2.2) -2,2-bis(4-hydroxyphenyl)propane;
20.0 parts (0.120 mol) of terephthalic acid;
4.3 parts (0.060 mol) of acrylic acid; and
0.5 part of titanium tetrabutoxide.

The flask was placed in a heating mantle equipped with a thermometer, a stirrer, a condenser, and a nitrogen inlet.

After the flask was purge with nitrogen gas, the contents of the flask were gradually heated with stirring and allowed to react with stirring at 200° C. for 4 hours (first reaction).

Then, 1.0 part (0.00 5mol) of trimellitic anhydride was added into the flask, and the contends of the flask were allowed to react at 180° C. for 1 hour (second reaction) to yield binder resin 3.

The acid value of binder resin 3 was 0 mg KOH/g, and the hydroxy value thereof was 82 mg KOH/g. Binder resin 3 was subjected to gel permeation chromatography (GPC) to measure the molecular weight. The weight average molecular weight (Mw) was 8,000; the number average molecular weight (Mn) was 3,500; and the peak molecular weight (Mp) was 5,700. Also, the softening point was 92° C.

### Preparation of Binder Resins 4 to 6

Binder resins 4 to 6 were prepared in the same manner as binder resin 3, except that trimellitic anhydride was added in the proportion shown in Table 2 to vary the acid value of the resulting binder resin. The acid value and hydroxy value of each of binder resins 4 to 6 are shown in Table 2.

### Preparation of Binder Resin 7

A four-neck flask was charged with the following ingredients:
80.00 parts of styrene;
20.00 parts of n-butyl acrylate; and
0.8 part of 2,2-bis(4,4-di-t-butylperoxycyclohexyl)propane.

The flask was sufficiently purged with nitrogen and heated to 130° C. while the contents of the flask were being stirred, and, then, 200 pats of xylene was dropped over a period of 3 hours. Xylene was refluxed for a polymerization reaction. After the completion of the polymerization, the solvent was removed by evaporation under reduced pressure to yield binder resin 9.

The acid value of the resulting binder resin 9 was lower than detection limit. The glass transition temperature Tg was 56° C. Binder resin 9 was subjected to gel permeation chromatography (GPC) to measure the molecular weight. The weight average molecular weight (Mw) was 50,000; the number average molecular weight (Mn) was 10,000; and the peak molecular weight (Mp) was 18,000. Also, the softening point was 108° C.

TABLE 2

| | Acid monomer of polyester resin | | | |
|---|---|---|---|---|
| | Terephthalic | adipic | Trimellitic | Acrylic |
| | acid | acid | anhydride | acid |
| | (parts) | (parts) | (parts) | (parts) |
| Binder resin 1 | 25.00 | 8.00 | 1.20 | — |
| Binder resin 2 | 24.10 | — | 5.80 | — |
| Binder resin 3 | 24.10 | — | 3.50 | 4.30 |
| Binder resin 4 | 24.10 | — | 4.70 | 4.30 |
| Binder resin 5 | 20.00 | — | 1.00 | 4.30 |
| Binder resin 6 | 24.10 | — | 5.30 | 4.30 |

| | Acid value | Hydroxyl value |
|---|---|---|
| | (mg KOH/g) | (mg KOH/g) |
| Binder resin 1 | 5.0 | 65.0 |
| Binder resin 2 | 15.0 | 12.0 |
| Binder resin 3 | 12.0 | 57.0 |
| Binder resin 4 | 18.0 | 54.0 |
| Binder resin 5 | 0.0 | 82.0 |
| Binder resin 6 | 22.0 | 56.5 |

### Preparation of Resin Composition 1

An autoclave was charged with the following ingredients:

18 parts of low-density polyethylene (Mw: 1,400, Mn: 850, DSC-measured highest endothermic peak.: 100° C.);
66 parts of styrene;
13.5 parts of n-butyl acrylate; and
2.5 parts of acrylonitrile.

After being purged with N₂, the reaction system was heated with stirring and kept at 180° C. Into the reaction system was successively dropped 50 parts of a solution of 2% by mass t-butyl hydroperoxide in xylene over a period of 5 hours. After cooling, the solvent was removed, yielding resin composition 1 containing a reaction product of the low-density polyethylene with a vinyl resin component.

The molecular weight of resin composition 1 was measured. The weight average molecular weight (Mw) was 7,100 and the number average molecular weight (Mn) was 3,000. Resin composition 1 was dispersed in 45% by volume methanol aqueous solution. The dispersion exhibited a transmittance of 69% at 25° C. for light having a wavelength of 600 nm.

### Preparation of Resin Composition 2

An autoclave was charged with the following ingredients:

20.0 parts of low-density polyethylene (Mw: 1,300, Mn: 800, DSC-measured highest endothermic peak: 95° C.);
65.0 parts of o-methylstyrene;
11.0 parts of n-butyl acrylate; and
4.0 parts of methacrylonitrile.

After being purged with. N₂, the reaction system was heated with stirring and kept at 170° C. Into the reaction system was successively dropped 50 parts of a solution of 2% by mass t-butyl hydroperoxide in xylene over a period of 5 hours. After cooling, the solvent was removed, yielding resin composition 2 containing a reaction product of the low-density polyethylene with a vinyl resin component.

The molecular weight of resin composition 2 was measured. The weight average molecular weight (Mw) was 6,900 and the number average molecular weight (Mn) was 2,900. Resin composition 2 was dispersed in 45% by volume methanol aqueous solution. The dispersion exhibited a transmittance of 63% at 25° C. for light having a wavelength of 600 nm.

### Preparation of Toner 1

### The Following Ingredients:

70.0 parts of binder resin 1;
30.0 parts of binder resin 2;
5.0 parts of Fischer-Tropsch wax (highest endothermic peak temperature: 90° C.);
3.0 parts of coloring agent 1, which dominantly contains a formula (1) compound;
6.0 parts of coloring agent 8;
0.5 part of aluminum 3,5-di-t-butylsalicylate; and
5.0 parts of resin composition 1
   were sufficiently mixed at a rotational speed of 20 s⁻¹ for 5 minutes using a Henschel mixer model FM-75 (manufactured by Nippon Coke & Engineering). The mixture was kneaded in a twin-screw kneader (PCM-30, manufactured by Ikegai) set at a temperature of 125° C. The kneaded product was cooled and roughly crushed to 1 mm or less with a hammer mill. The roughly crushed product was further pulverized to a much lower particle size with a mechanical pulverizer (T-250, manufactured by Turbo Kogyo). Furthermore, the pulverized product was classified with a rotational classifier 200TSP (manufactured by Hosokawa Micron) to yield toner particles. The rotational classifier 200TSP was operated at classification rotor rotational speed of 50.0 s⁻¹. The weight-average particle size (D4) of the resulting toner particles was 6.2 μm.

To 100 parts of the toner particles were added 0.8 part of hydrophobic silica fine particles surface-treated with 20% by mass of hexamethyldisilazane and. having a number average primary particle size of 10 nm, and 0.2 part of titanium oxide fine particles surface-treated with 16% by mass of isobutyl(trimethoxy)silane and having a number average primary particle size of 30 nm. The ingredients were mixed with a Henschel mixer model FM-75 (manufactured by Nippon Coke & Engineering) at a rotational speed of 30 s⁻¹ for 10 minutes to yield toner 1. The spectral reflectance of toner 1 is shown in. Table 3.

### Preparation of Toners 2 to 8 and 27 to 29

Toners 2 to 8 and 27 to 29 were prepared in the same manner as toner 1 except that the binder resins, the wax, the resin composition, the coloring agents, and the proportions thereof were replaced with those shown in Table 3.

### Preparation of Toner 9

A 2 L four-neck flask equipped with a high-speed stirrer Clearmix (manufactured by M Technique) was charged with 470 parts of ion-exchanged water and 3.3 parts of Na₃PO₄, and the contents of the flask were heated to 65° C. with the stirrer set at a rotational speed of 10,000 rpm. A CaCl₂ aqueous solution was added into the flask to prepare an aqueous dispersion medium containing very small particles of a poorly water-soluble dispersant Ca₃(PO₄)₂.

A mixture of the following ingredients was prepared as dispersoid:
100.0 parts of binder resin 7;
5.0 parts of ester wax (highest endothermic peak temperature: 100° C.);
3.0 parts of coloring agent 1, which dominantly contains formula (1) compound;
6.0 parts of coloring agent 9; and
0.5 part of aluminum 3,5-di-t-butylsalicylate.

This mixture was agitated in an attritor (manufactured by Nippon Coke & Engineering) for 3 hours, and 3 parts of 2,2′-azobis(2,4-dimethylvaleronitrile) was added to the mixture at 65° C., followed by stirring for 1 minute to yield a polymerizable monomer composition. The resulting polymerizable monomer composition was added into the aqueous dispersion medium being stirred with a high-speed stirrer at a rotational speed of 15,000 rpm, and the contents of the reaction system were stirred for 3 minutes at 60° C. in a N2 atmosphere to granulate the polymerizable monomer composition. Then, the stirrer was replaced with another one with a stirring paddle, and the contents of the reaction system were kept at that temperature while being stirred at 200 rpm. When the percentage of polymer converted from the polymerizable vinyl-based monomer had reached 90%, the first reaction was completed. The reaction system was further heated to 80° C. for a second reaction. When the percentage of polymer converted from the monomer had reached about 100%, the second reaction was completed, and the entire polymerization process was completed. After completing the polymerization and cooling the reaction system, dilute hydrochloric acid was added to dissolve the poorly water-soluble dispersant. The polymerization product was rinsed with a pressure filter several times and dried to yield polymer particles. The weight-average particle size of the resulting polymer particles was 7.2 μm.

To 100 parts of the polymer particles were added 0.8 part of hydrophobic silica fine particles surface-treated with 20% by mass of hexamethyldisilazane and having a number average primary particle size of 10 nm, and 0.2 part of titanium oxide fine particles surface-treated with 16% by mass of isobutyl(trimethoxy)silane and having a number average primary particle size of 30 nm. The ingredients were mixed with a Henschel mixer model FM-75 (manufactured by Nippon Coke & Engineering) at a rotational speed of 30 s⁻¹ for 10 minutes to yield toner 9.

### Preparation of Toners 10 to 26

Toners 10 to 26 were prepared in the same manner as toner 9 except that the binder resin, the wax, the resin composition, the coloring agents, and the proportions thereof were replaced with those shown in Table 3.

TABLE 3

| | Binder resin (1) | | Binder resin (2) | | Wax | | Resin composition | |
|---|---|---|---|---|---|---|---|---|
| | | parts | | parts | | parts | | parts |
| Toner 1 | Binder resin 1 | 70 | Binder resin 2 | 30 | Fischer- | 5 | Resin composition 1 | 5 |
| Toner 2 | Binder resin 1 | 70 | Binder resin 2 | 30 | Tropsch | 5 | Resin composition 1 | 5 |
| Toner 3 | Binder resin 1 | 70 | Binder resin 2 | 30 | wax | 5 | Resin composition 1 | 5 |
| Toner 4 | Binder resin 3 | 70 | Binder resin 2 | 30 | Paraffin | 5 | Resin composition 1 | 5 |
| Toner 5 | Binder resin 4 | 70 | Binder resin 2 | 30 | wax | 5 | Resin composition 2 | 5 |
| Toner 6 | Binder resin 5 | 70 | Binder resin 2 | 30 | | 5 | Resin composition 2 | 5 |
| Toner 7 | Binder resin 6 | 70 | Binder resin 2 | 30 | Ester | 5 | Resin composition 2 | 5 |
| Toner 8 | Binder resin 6 | 70 | Binder resin 2 | 30 | wax | 5 | — | |
| Toner 9 | Binder resin 7 | 100 | — | | 5 | — | | |
| Toner 10 | Binder resin 7 | 100 | — | | 5 | — | | |
| Toner 11 | Binder resin 7 | 100 | — | | 5 | — | | |
| Toner 12 | Binder resin 7 | 100 | — | | 5 | — | | |
| Toner 13 | Binder resin 7 | 100 | — | | 5 | — | | |
| Toner 14 | Binder resin 7 | 100 | — | | 5 | — | | |
| Toner 15 | Binder resin 7 | 100 | — | | 5 | — | | |
| Toner 16 | Binder resin 7 | 100 | — | | 5 | — | | |
| Toner 17 | Binder resin 7 | 100 | — | | 5 | — | | |
| Toner 18 | Binder resin 7 | 100 | — | | 5 | — | | |
| Toner 19 | Binder resin 7 | 100 | — | | 5 | — | | |
| Toner 20 | Binder resin 7 | 100 | — | | 5 | — | | |
| Toner 21 | Binder resin 7 | 100 | — | | 5 | — | | |
| Toner 22 | Binder resin 7 | 100 | — | | 5 | — | | |
| Toner 23 | Binder resin 7 | 100 | — | | 5 | — | | |
| Toner 24 | Binder resin 7 | 100 | — | | 5 | — | | |
| Toner 25 | Binder resin 7 | 100 | — | | 5 | — | | |
| Toner 26 | Binder resin 7 | 100 | — | | 5 | — | | |
| Toner 27 | Binder resin 3 | 70 | Binder resin 2 | 30 | Fischer- | 5 | Resin composition 1 | 5 |
| Toner 28 | Binder resin 3 | 70 | Binder resin 2 | 30 | Tropsch | 5 | Resin composition 1 | 5 |
| Toner 29 | Binder resin 3 | 70 | Binder resin 2 | 30 | wax | 5 | Resin composition 1 | 5 |

| | Formula (1) compound | Coloring agent (1) | | Coloring agent (2) | |
|---|---|---|---|---|---|
| | parts | | parts | | parts |
| Toner 1 | 3 | Coloring agent 1 | 3 | Coloring agent 8 | 6 |
| Toner 2 | 3 | Coloring agent 1 | 3 | Coloring agent 9 | 6 |
| Toner 3 | 3 | Coloring agent 1 | 3 | Coloring agent 10 | 6 |
| Toner 4 | 3 | Coloring agent 1 | 3 | Coloring agent 8 | 6 |
| Toner 5 | 3 | Coloring agent 1 | 3 | Coloring agent 8 | 6 |
| Toner 6 | 3 | Coloring agent 1 | 3 | Coloring agent 8 | 6 |
| Toner 7 | 3 | Coloring agent 1 | 3 | Coloring agent 8 | 6 |
| Toner 8 | 3 | Coloring agent 1 | 3 | Coloring agent 8 | 6 |
| Toner 9 | 3 | Coloring agent 1 | 3 | Coloring agent 9 | 6 |
| Toner 10 | 3 | Coloring agent 1 | 3 | Coloring agent 9 | 6 |
| Toner 11 | 3 | Coloring agent 1 | 3 | Coloring agent 9 | 6 |
| Toner 12 | 3 | Coloring agent 1 | 3 | Coloring agent 10 | 6 |
| Toner 13 | 3 | Coloring agent 1 | 3 | Coloring agent 10 | 6 |
| Toner 14 | 3 | Coloring agent 1 | 3 | Coloring agent 10 | 6 |
| Toner 15 | 3 | Coloring agent 1 | 3 | — | |
| Toner 16 | 4 | Coloring agent 1 | 4 | — | |
| Toner 17 | 10 | Coloring agent 1 | 10 | — | |
| Toner 18 | 2 | Coloring agent 1 | 2 | — | |
| Toner 19 | 15 | Coloring agent 1 | 15 | — | |
| Toner 20 | 0.5 | Coloring agent 1 | 0.5 | — | |
| Toner 21 | 22 | Coloring agent 1 | 22 | — | |
| Toner 22 | 22 | Coloring agent 2 | 22 | — | |
| Toner 23 | 22 | Coloring agent 4 | 22 | — | |
| Toner 24 | 22 | Coloring agent 3 | 22 | — | |
| Toner 25 | 22 | Coloring agent 5 | 22 | — | |
| Toner 26 | 22 | Coloring agent 6 | 22 | — | |
| Toner 27 | 3 | Coloring agent 7 | 3 | Coloring agent 10 | 6 |
| Toner 28 | — | Pigment Red 150.31 | 3 | Coloring agent 10 | 6 |
| Toner 29 | — | Pigment Red 150.31 | 3 | Coloring agent 10 | 6 |

| | Spectral reflectance (%) of toner | |
|---|---|---|
| | 400-500 nm | 650-700 nm |
| Toner 1 | 10 | 95 |
| Toner 2 | 10 | 95 |
| Toner 3 | 10 | 95 |
| Toner 4 | 10 | 95 |
| Toner 5 | 10 | 95 |
| Toner 6 | 10 | 95 |
| Toner 7 | 10 | 95 |
| Toner 8 | 10 | 95 |
| Toner 9 | 10 | 95 |
| Toner 10 | 15 | 93 |
| Toner 11 | 15 | 93 |
| Toner 12 | 23 | 90 |
| Toner 13 | 23 | 90 |
| Toner 14 | 15 | 85 |
| Toner 15 | 30 | 85 |
| Toner 16 | 30 | 85 |
| Toner 17 | 30 | 85 |
| Toner 18 | 30 | 85 |
| Toner 19 | 30 | 85 |
| Toner 20 | 30 | 85 |
| Toner 21 | 30 | 85 |
| Toner 22 | 30 | 85 |
| Toner 23 | 30 | 85 |
| Toner 24 | 30 | 85 |
| Toner 25 | 30 | 85 |
| Toner 26 | 30 | 85 |
| Toner 27 | 14 | 93 |
| Toner 28 | 14 | 93 |
| Toner 29 | 14 | 93 |

### Preparation of Magnetic Carrier

Water was added to 100 parts of Fe₂O₃, and the Fe₂O₃ was crushed for 15 minutes in a ball mill to yield magnetic core particles having an average particle size of 55 μm.

Subsequently, the mixture of I part of straight silicone resin KR271 (produced by Shin-Etsu. Chemical), 0.5 part of γ-aminopropyltriethoxysilane, and 98.5 parts of toluene was added to 100 parts of the magnetic core particles. While the mixture was being stirred in a solution decompression kneader, the solvent was removed by drying under reduced pressure at 70° C. for 5 hours. Then, the contents in the kneader were fired at 140° C. for 2 hours and was sieved with a sieve shaker model 300 MM-2 (75 μm openings, manufactured by Tsutsui Science Instruments) to yield magnetic carrier 1.

### Examples 1 to 26, Comparative Examples 1 to 3

Two-component developer 1 was prepared by mixing toner 1 and magnetic carrier 1 with a toner content of 9% by mass at a rotational speed of 0.5 s⁻¹ for 5 minutes with a mixer model V-10 (manufactured by Tokuju Corporation). Two-component developers 2 to 29 were prepared with respective combinations of a toner and a magnetic carrier shown Table 4. Then, the two-component developers of Examples 1 to 26 and Comparative Examples 1 to 3 were examined for evaluation as below. Examination results of Examples 1 to 26 and Comparative Examples 1 to 3 are shown in Table 5.

TABLE 4

| | Toner | Carrier | Two-component developer |
|---|---|---|---|
| Example 1 | Toner 1 | Carrier 1 | Two-component developer 1 |
| Example 2 | Toner 2 | Carrier 1 | Two-component developer 2 |
| Example 3 | Toner 3 | Carrier 1 | Two-component developer 3 |
| Example 4 | Toner 4 | Carrier 1 | Two-component developer 4 |
| Example 5 | Toner 5 | Carrier 1 | Two-component developer 5 |
| Example 6 | Toner 6 | Carrier 1 | Two-component developer 6 |
| Example 7 | Toner 7 | Carrier 1 | Two-component developer 7 |
| Example 8 | Toner 8 | Carrier 1 | Two-component developer 8 |
| Example 9 | Toner 9 | Carrier 1 | Two-component developer 9 |
| Example 10 | Toner 10 | Carrier 1 | Two-component developer 10 |
| Example 11 | Toner 11 | Carrier 1 | Two-component developer 11 |
| Example 12 | Toner 12 | Carrier 1 | Two-component developer 12 |
| Example 13 | Toner 13 | Carrier 1 | Two-component developer 13 |
| Example 14 | Toner 14 | Carrier 1 | Two-component developer 14 |
| Example 15 | Toner 15 | Carrier 1 | Two-component developer 15 |
| Example 16 | Toner 16 | Carrier 1 | Two-component developer 16 |
| Example 17 | Toner 17 | Carrier 1 | Two-component developer 17 |
| Example 18 | Toner 18 | Carrier 1 | Two-component developer 18 |
| Example 19 | Toner 19 | Carrier 1 | Two-component developer 19 |
| Example 20 | Toner 20 | Carrier 1 | Two-component developer 20 |
| Example 21 | Toner 21 | Carrier 1 | Two-component developer 21 |
| Example 22 | Toner 22 | Carrier 1 | Two-component developer 22 |
| Example 23 | Toner 23 | Carrier 1 | Two-component developer 23 |
| Example 24 | Toner 24 | Carrier 1 | Two-component developer 24 |
| Example 25 | Toner 25 | Carrier 1 | Two-component developer 25 |
| Example 26 | Toner 26 | Carrier 1 | Two-component developer 26 |
| Comparative | Toner 27 | Carrier 1 | Two-component developer 27 |
| Example 1 | | | |
| Comparative | Toner 28 | Carrier 1 | Two-component developer 28 |
| Example 2 | | | |
| Comparative | Toner 29 | Carrier 1 | Two-component developer 29 |
| Example 3 | | | |

Examination of Toner Tinting strength

A copy machine modified from a full color copy machine, image RUNNER ADVANCE C5255 (manufactured by Canon) was used as an electrophotographic image forming apparatus, and the developing unit of the magenta station was charged with the corresponding two-component developer shown in Table 4.

The examination was performed in an environment of normal temperature and normal humidity (23° C., 50% RH), using plain copy paper sheets, GFC-081 (A4, basis weight: 81.4 g/m², available from Canon Marketing Japan).

First, the amount of toner to be deposited on the paper was varied, and the relationship between the image density and the amount of toner on the paper was examined.

Subsequently, the copy machine was adjusted so that the image density of an FFH pattern (solid pattern) could be 1.40, and the amount of toner on the paper when the image density was 1.40 was determined.

FFH refers to a value of 256 gradations represented in hexadecimal notation; 00H represents the first gradation (blank) of the 256 gradations; and FFH represents the 256th gradation (solid) of the 256 gradations.

The image density was measured with one of color reflection densitometer 500 series (manufactured by X-Rite).

The tinting strength of each toner was rated by the amount (mg/cm²) of toner deposited on the paper according to the following criteria. The results are shown in Table 5.

### Criteria:

A: less than 0.35 (excellent)
B: 0.35 to less than. 0.50 (good)
C: 0.50 to less than 0.65 (acceptable in view of the disclosure)
D: 0.65 or more (not acceptable)

### Examination of Color Change

A copy machine modified from a full color copy machine, image RUNNER ADVANCE C5255 (manufactured by Canon) was used as an electrophotographic image forming apparatus, and the developing unit of the magenta station was charged with the corresponding two-component developer shown in Table 4.

The examination was performed in an environment of 20° C. and 8% RH, using plain copy paper sheets, GIF-081 (A4, basis weight: 81.4 g/m², available from Canon Marketing Japan).

A 16-gradation pattern was formed by varying the amount of toner deposited. The L*, a*, and b* values of the resulting pattern were measured at a D50 viewing angle of 2° with Spectra Scan Transmission (manufactured by Gretag Macbeth). In the measurement, the L1*, a1*, and b1* values at an amount of toner at which C* became 85 in the T*-c* coordinate system were measured.

Subsequently, the copy machine was adjusted so that the image density of an FFH pattern (solid pattern) could be 1.40, and the amount of Loner on the paper when the image density was 1.45 was determined for adjusting the developing bias.

After adjusting the developing bias, a pattern with a coverage of 1% was printed on 50,000 sheets of paper while the toner was fed so that the toner density could be constant.

After outputting 50,000 sheets, a 16-gradation pattern was formed by varying the amount of toner deposited. The L*, a*, and b* values of the resulting pattern were measured at a D50 viewing angle of 2° with Spectra Scan Transmission (manufactured by Gretag Macbeth). In the measurement, the L2*, a2*, and b2* values at an amount of toner at which became 85 in the L*-c* coordinate system were measured, and ΔE was calculated from the L*, a*, and b* values of the patterns at the beginning of the examination and after outputting 50,000 sheets. The results are shown in Table 5.

ΔE={(L1*−L2*)2+(a1*−a2*)2+(b1*−b2*)2}1/2
A: ΔE was so low as color change was not visually detectable.
B. ΔE was higher than that in the case rated as A, but color change was not visually detectable.
C: ΔE was higher than that in the case rated as B, but color change was little detectable visually.
D: ΔE was higher than that in the case rated as C and color change was visually detectable.
   Fogging over Blank Portion (Portion Having No Pattern)

A copy machine modified from a full color copy machine, image RUNNER ADVANCE C5255 (manufactured by Canon) was used as an electrophotographic image forming apparatus, and the developing unit of the magenta station was charged with the corresponding two-component developer shown in Table 4.

The examination was performed in an environment of normal temperature and normal humidity (23° C., 50% RH), using plain copy paper sheets, GFC-081 (A4, basis weight: 81.4 g/m², available from Canon Marketing Japan).

The fogging over the black portion on the first sheet and the 50,000th sheet was measured.

The average reflectance Dr (%) of the test paper sheet before image output was measured with a reflectometer model TC-6DS (manufactured by Tokyo Denshoku).

The reflectance Ds (%) of the 00H pattern (blank portion) on the first sheet and the 50,000th sheet was measured. Fogging (%) was calculated from. the obtained Dr value and Ds value (for the first and the 50,000th) by the equation: fogging (%)=Dr (%)−Ds (%). The fogging was rated according to the following criteria

The results are shown in Table 5.

### Criteria:

A: The fogging value was low, and fogging was not visually detectable.
B: The fogging value was higher than that in the case rated as A, but fogging was not visually detectable.
C: The fogging value was higher than that in the case rated. as B, but fogging was little detectable visually.
D: The fogging value was higher than that in the case rated as C and fogging was visually detectable.

TABLE 5

| | Tinting strength | | | | | | | |
|---|---|---|---|---|---|---|---|---|
| | Amount of toner deposited for | | Color | | Fogging | | | |
| | image density of 1.40 | | change | | First | | 50,000th | |
| | (mg/cm²) | Grade | ΔE | Grade | (%) | Grade | (%) | Grade |
| Example 1 | 0.32 | A | 0.2 | A | 0.1 | A | 0.2 | A |
| Example 2 | 0.33 | A | 0.4 | A | 0.1 | A | 0.2 | A |
| Example 3 | 0.32 | A | 0.7 | A | 0.2 | A | 0.2 | A |
| Example 4 | 0.33 | A | 1.3 | A | 0.2 | A | 0.2 | A |
| Example 5 | 0.32 | A | 1.4 | A | 0.2 | A | 0.2 | A |
| Example 6 | 0.33 | A | 1.5 | A | 0.2 | A | 0.3 | A |
| Example 7 | 0.34 | A | 2.0 | B | 0.3 | A | 0.4 | A |
| Example 8 | 0.34 | A | 2.3 | B | 0.5 | B | 0.5 | B |
| Example 9 | 0.34 | A | 2.3 | B | 0.3 | A | 0.4 | A |
| Example 10 | 0.34 | A | 2.5 | B | 0.3 | A | 0.4 | A |
| Example 11 | 0.34 | A | 2.4 | B | 0.3 | A | 0.4 | A |
| Example 12 | 0.36 | B | 2.3 | B | 0.3 | A | 0.4 | A |
| Example 13 | 0.35 | B | 2.4 | B | 0.3 | A | 0.4 | A |
| Example 14 | 0.38 | B | 2.5 | B | 0.3 | A | 0.6 | B |
| Example 15 | 0.40 | B | 2.7 | B | 0.3 | A | 0.7 | B |
| Example 16 | 0.40 | B | 2.7 | B | 0.3 | A | 0.7 | B |
| Example 17 | 0.43 | B | 2.8 | B | 0.3 | A | 0.7 | B |
| Example 18 | 0.45 | B | 2.9 | B | 0.3 | A | 0.7 | B |
| Example 19 | 0.42 | B | 2.7 | B | 0.3 | A | 0.8 | B |
| Example 20 | 0.42 | B | 2.9 | B | 0.4 | A | 0.8 | B |
| Example 21 | 0.41 | B | 3.0 | B | 0.3 | A | 0.8 | B |
| Example 22 | 0.43 | B | 3.1 | B | 0.3 | A | 0.8 | B |
| Example 23 | 0.46 | B | 3.0 | B | 0.4 | A | 0.8 | B |
| Example 24 | 0.44 | B | 3.1 | B | 0.3 | A | 0.8 | B |
| Example 25 | 0.49 | B | 3.2 | B | 0.4 | A | 0.7 | B |
| Example 26 | 0.41 | B | 3.1 | B | 0.4 | A | 0.9 | B |
| Comparative | 0.49 | B | 3.6 | C | 1.0 | C | 1.5 | C |
| Example 1 | | | | | | | | |
| Comparative | 0.49 | B | 4.0 | C | 1.1 | C | 1.4 | C |
| Example 2 | | | | | | | | |
| Comparative | 0.59 | C | 4.2 | C | 0.8 | B | 1.3 | C |
| Example 3 | | | | | | | | |

While the present disclosure has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-035395 filed Feb. 27, 2017, which is hereby incorporated by reference herein in its entirety.

What is claimed is:

1. A toner comprising: toner particles, each containing a binder resin and a crystalline compound exhibiting a CuKα X-ray diffraction spectrum having a diffraction peak at a Bragg angle 2θ±0.2 in the range of 4.0° to 5.0°, the diffraction peak having a half-value width of 0.7° or more, the crystalline compound being represented by formula (1): 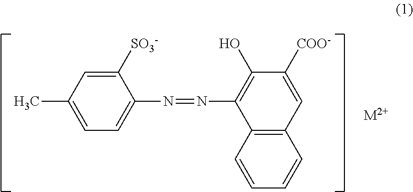
wherein M represents an atom selected from the group consisting of barium, strontium, calcium, and manganese.

2. The toner according to claim 1, wherein the diffraction peak at a Bragg angle 2θ±0.2 in the range of 4.0° to 5.0° has a half-value width in the range of 0.7° to 1.5°.

3. The toner according to claim 1, wherein the proportion of the compound represented by formula (1) in the toner is in the range of 1.0 part by mass to 20.0 parts by mass relative to 100 parts by mass of the binder resin.

4. The toner according to claim 1, wherein the toner particle further contains a quinacridone pigment.

5. The toner according to claim 1, wherein the toner has a reflectance of 25% or less for a wavelength in the range of 400 nm to 500 nm and a reflectance of 90% or more for a wavelength in the range of 650 nm to 700 nm.

6. The toner according to claim 1, wherein the binder resin is a polyester resin.

7. The toner according to claim 6, wherein the polyester resin has an acid value in the range of 0 mg KOH/g to 20 mg KOH/g.
